(19) United States
(12) Patent Application Publication
(10) Pub. No.: US 2026/0157709 A1
(43) Pub. Date: Jun. 11, 2026

(54) METHOD FOR POSITIONAL ASSIGNMENT OF A PET ATTENUATION MAP OF A MOVABLE OBJECT

(71) Applicant: Siemens Healthineers AG, Forchheim (DE)

(72) Inventors: Johann SUKKAU, Herzogenaurach (DE); Manuel SCHNEIDER, Unterfoehring (DE); Matthias FENCHEL, Bubenreuth (DE)

(73) Assignee: Siemens Healthineers AG, Forchheim (DE)

(21) Appl. No.: 19/408,602

(22) Filed: Dec. 4, 2025

(30) Foreign Application Priority Data
Dec. 6, 2024 (DE) 10 2024 211 681.5

(51) Int. Cl. A61B 6/04; A61B 6/00; A61B 6/58

(52) U.S. Cl. CPC A61B 6/0407; A61B 6/5235; A61B 6/547; A61B 6/584

(57) ABSTRACT

One or more example embodiments relates to a method for positional assignment of a PET attenuation map of a movable object relative to a movable patient couch of an MR-PET system.

### CROSS-REFERENCE TO RELATED APPLICATION(S)

The present application claims priority under 35 U.S.C. § 119 to Germany Patent Application No. 10 2024 211 681.5, filed Dec. 6, 2024, the entire contents of which is incorporated herein by reference.

### FIELD

One or more example embodiments relates to a method and an assignment system for positional assignment of a positron emission tomography attenuation map of a movable object and to a corresponding hybrid system.

Independent of the grammatical term usage, individuals with male, female or other gender identities are included within the term.

### RELATED ART

Positron Emission Tomography devices (PET devices) are imaging apparatuses of nuclear medicine, which use Gamma-F rays or Gamma quanta for imaging biochemical and physiological functions that arise after the decay of a radio nucleoid emitting positrons. These Gamma quanta are detected so that the distribution of a weakly radioactive substance in the organism can be displayed visually in the form of slice images. For localization of the radioactive substance in the organism the PET method is as a rule carried out as a hybrid method together with Computed Tomography (CT) or Magnetic Resonance Tomography (MRT). According to the current prior art the Gamma photons are further converted in particular via LSO crystals into Gamma photons and via CMOS photodetectors into electrons. The Gamma quanta in a PET scanner are slowed down by material that is located between the point at which the Gamma quanta arise through annihilation and the Gamma detectors. This attenuation by the material can have a negative influence on the PET signal quality and it is therefore an object to correct this attenuation retroactively, in as far as it cannot be avoided, (for example by obstacles being removed on the path to the detectors). PET attenuation maps, so-called μ maps, which are available by default for installed equipment such as the patient couch, the local MR coils and the PET phantoms are employed for retroactive correction of the PET signals. The μ maps are calculated for example with data recorded by the CT or the MR as LAC (Linear Attention Coefficient) values. In this case the recorded data is especially stored as 3-dimensional voxel data.

The μ maps can then be used in order to correct the measurement data during subsequent measurements with the PET device. This is referred to as Attenuation Correction (AC). A challenge when using the attenuation maps is a positioning of components able to be freely positioned, at least in part, in the measurement area, such as for example of PET phantoms, relative to the patient couch and thus also relative to the measurement system. The position of the respective components is needed however in order to accurately carry out the attenuation correction with the aid of the attenuation maps. In particular it must be established where the respective component is located relative to an attenuation source and/or to PET measurement data.

In the prior art the approach is therefore that of letting a user determine the position of the phantom manually. For example the user can overlay two images, namely a PET image of the respective component and the attenuation map in a user interface until the images are adequately congruent.

### SUMMARY

This method is time-consuming for the user however.

One or more example embodiments therefore provides a solution with which a positioning of a movable object relative to a PET attenuation source is made possible automatically. In particular it is also optional to precisely determine the position of a PET phantom relative to a patient table and/or relative to a measurement system of a PET hybrid system.

### BRIEF DESCRIPTION OF THE DRAWINGS

Advantages and characteristic features of exampble embodiments emerge from the description given below which refers to the enclosed figures. In the figures, features that are the same are used with the same reference characters in each case. It goes without saying here that individual features that are explicitly described for a specific form of embodiment can also find their way into other forms of embodiment of the invention for use, provided this is not forbidden because of technical circumstances.

### In the Figures:

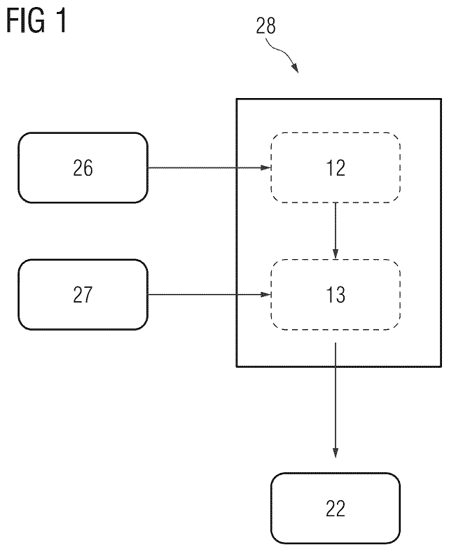

FIG. 1 shows a flow diagram of a method for

positional assignment of a PET attenuation map of a movable object in accordance with one form of embodiment of the invention,

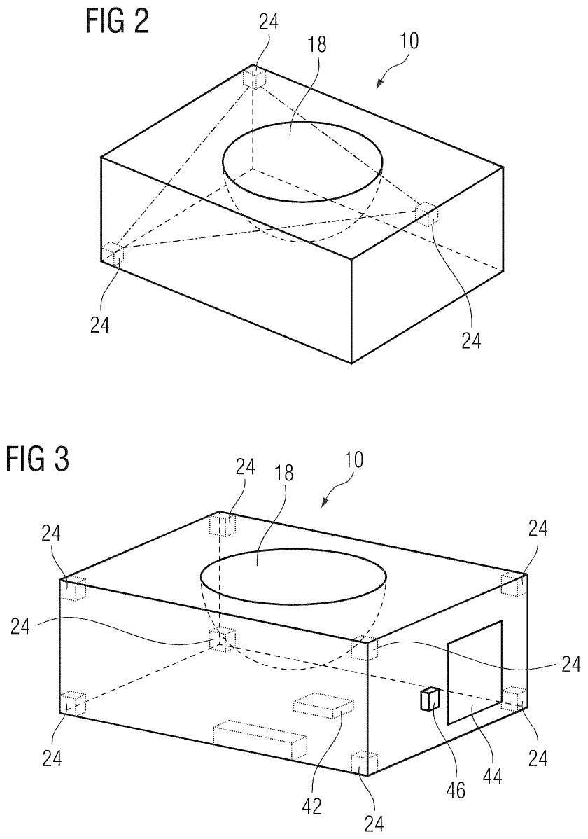

FIG. 2 shows a perspective view of an inventive form of embodiment of a phantom holder with three magnetic field strength sensors,

FIG. 3 shows a perspective view of an inventive form of embodiment of a phantom holder with eight magnetic field strength sensors, and

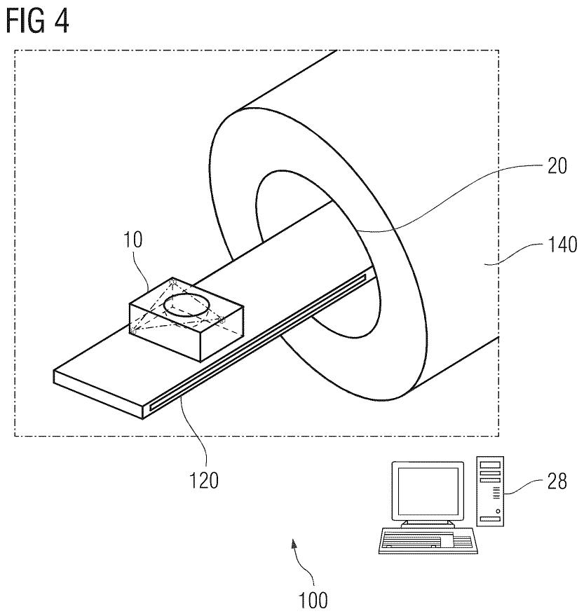

FIG. 4 shows a perspective view of an inventive form of embodiment of a hybrid system for magnetic resonance tomography and positron emission tomography.

### DETAILED DESCRIPTION

In accordance with one or more example embodiments, a method for positional assignment of a Positron Emission Tomography attenuation map, PET attenuation map, of a movable object, in particular of a magnetic resonance tomography accessory part, relative to a movable patient couch of a hybrid system for magnetic resonance tomography and Positron Emission Tomography, MR-PET, is provided, wherein the MR-PET comprises the movable patient couch and a patient tunnel with a B0 main magnet, wherein the position and/or orientation of the object with respect to at least one degree of freedom is predetermined by the construction and with respect to at least one further degree of freedom is free, wherein the object comprises at least one magnetic field sensor, in particular one magnetic field sensor per non-predetermined degree of freedom, at a respective defined relative position, wherein the method comprises the following steps: positioning of the movable object within a stray magnetic field of the B0 main magnet on the patient couch when the patient couch is positioned outside the patient tunnel; recording of magnetic field measurement data with the at least one magnetic field sensor and transfer of the magnetic field measurement data to an evaluation apparatus, wherein information about the at least one constructionally predetermined degree of freedom is transferred to the evaluation apparatus or wherein this information is stored on the evaluation apparatus and in particular an object ID for assignment of the object (10) to the information is transferred to the evaluation apparatus; optionally: transfer of the position of the patient couch to the evaluation apparatus; calculation of first object position data of the object relative to the patient couch and/or to the main magnet with the evaluation apparatus based on the magnetic field measurement data, optionally on the position of the patient couch and on the information about the at least one constructionally predetermined degree of freedom; movement of the patient couch with the movable object into the patient tunnel and transfer of the moved-in position and/or of the change of position of the patient couch to the evaluation apparatus; calculation of second object position data relative to the B0 main magnet with the evaluation apparatus based on the first object position data and the moved-in position and/or the change of position of patient couch and determination of the position of the PET attenuation map and/or selection of a section of the PET attenuation map based on the second object positioning data. What is inventive about the method is that the movable object is defined in at least one direction of movement, so that the degrees of freedom are restricted in at least one direction. Typically an object has a number of degrees of freedom, in particular comprising degrees of freedom of rotation (concerning an orientation of the object) and degrees of translation freedom (concerning a position, in particular in three spatial directions). The freedom of movement of the object can be partly restricted by outside circumstances, however. For example a height of the object can be predetermined by the object being laid on the patient couch, which has a fixed height. Within the framework of example embodiments this is described by the constructionally predetermined degrees of freedom. If degrees of freedom are constructionally predetermined, then, in the intended use of the object in the hybrid system, they are in particular no longer free, but fixed. Advantageously, with at least one fixed degree of freedom, a more exact positioning and also a more exact position determination can be made possible than if all degrees of freedom were free. For example an orientation of the object can be fixed, in particular by a constructional match between the object and the patient couch. For example a height of the object can be fixed, in particular by the object being laid directly on the patient couch and/or on a fixed component of the patient couch. Likewise there is provision for at least one degree of freedom to remain in place, for example the possibility of movement in the Z direction. The more open degrees of freedom the movable object has, the more difficult the precise position determination relative to the patient couch tends to be. However it is not always practicable or indeed possible for all degrees of freedom to be predetermined as fixed or to be fixed with a sufficient precision. Here the inventive method is advantageous in that the missing (free or not fixed) degrees of freedom can be established automatically and thus in particular a precise position determination is possible. The inventive method thus on the one hand utilizes the precise positioning with the aid of the constructionally predetermined degrees of freedom and on the other hand allows a position determination of the remaining degrees of freedom.

The Z direction or the Z axis is generally understood as the direction along the axis of the patient tunnel and/or the direction in which the patient couch is moved into or is moved out of the patient tunnel. The X direction/axis and Y direction/axis are the directions that run at right angles to the Z direction. In particular the X direction typically runs horizontally and the Y direction typically runs vertically. The X axis is often that axis along which, within the framework of an MR measurement, a frequency encoding takes place, and the Y axis is often that axis along which, within the framework of an MR measurement, a phase encoding takes place. The X, Y, Z axes together can represent a coordinate system of the hybrid system of the main magnet. When, within the framework of example embodiments, reference is made to a coordinate system, this is the coordinate system that is typically meant. The X, Y and Z direction can be referred to as coordinate directions of the coordinate system of the hybrid system or of the main magnet. In addition there is also the coordinate system of the patient couch, the origin of which is defined with the aid of the position of the patient couch. Typically the coordinate system of the patient couch is offset from the coordinate system of the main magnet in the Z direction, wherein the offset depends on the position of the patient couch. For reasons involving the method it can sometimes also be advantageous to also define a PET coordinate system, which describes the PET system of the hybrid system. A conversion of a position in the coordinate system of the main magnet into the PET coordinate system can take place for example with the aid of a table offset matrix known in the prior art.

The movable object is preferably a magnetic resonance tomography accessory part and/or an accessory part of the hybrid system. For example the magnetic resonance tomography accessory part can be a local MR coil, which is positioned on a part of the body of a patient to be examined. In particular the breast coil is usually only able to be moved in the Z direction in this case. In particular the knee coil is able to be moved in the Z and in the X direction, but not able to be moved in the Y direction and is not rotatable. By contrast for example shoulder coils are usually wound multidimensionally around the shoulder of the patient, so that the coordinates of the associated MR coil are often not able to be fixed by default relative to the patient couch. For example the movable object can be a phantom, in particular PET phantom.

The magnetic field sensor can be a three-dimensional (3D) magnetic field sensor. The magnetic field sensor can in particular be a Hall sensor or a 3D Hall sensor. A three-dimensional magnetic field sensor is in particular a magnetic field sensor that can detect a magnetic field strength for each of three spatial directions. For example three Hall elements can be provided, which can each determine a magnetic field strength in one spatial direction. For example the magnetic field measurement data can comprise the magnetic field strengths in three spatial directions. For each open degree of freedom, for position determination of the movable object, one magnetic field sensor in particular can be provided. In a simplest case, for example in some cases during use of a knee or breast coil, there can be provision for just one magnetic field sensor to be used. Preferably two sensors can be used for a knee coil, in particular in order to take account of two degrees of freedom of the knee coil, in particular in the Z and X direction.

In a first method step the movable object is positioned within a stray magnetic field of the B0 main magnet on the patient couch when the patient couch is located outside the patient tunnel. The stray magnetic field can in particular be understood as the magnetic field outside of the patient tunnel or outside of the main magnet of the magnetic resonance tomography system. The stray field can in particular be the stray field of the B0 magnetic field of the magnetic resonance tomography system or of the main magnet of the magnetic resonance tomography system or comprise said field. While the magnetic field in the main magnet is largely homogeneous, this magnetic field falls outside of the main magnet (in the area of the stray field). Basically the magnetic field is as a rule all the smaller the further the distance from the patient tunnel is. Thus with the help of information about the course of the magnetic field outside of the patient tunnel, in conjunction with the measured stray field, the position of the magnetic field sensor can be deduced. In this arrangement first magnetic field measurement data, in particular from the plurality of magnetic field sensors corresponding to the number of open degrees of freedom, is recorded and is transferred to an evaluation apparatus, by which it is in particular received and/or stored. The evaluation apparatus can for example be part of a control console of the hybrid system. In particular the evaluation apparatus can comprise a processor unit for processing and calculation of the magnetic field data. The magnetic field measurement data can in particular be transferred with the aid of a transfer means to the evaluation apparatus. The measurement data is transmitted to the evaluation apparatus wirelessly and/or by a cable. The transmission means can for example be a communication apparatus, in particular comprising a transmitter and a receiver, which has a communication link to the evaluation apparatus. The transmitter can in particular be arranged and/or fixed on the object. Optionally a wireless transmission between the communication apparatus and the receiver can be provided. First object position data relative to the B0 main magnet is calculated from the magnetic field measurement data, which is based on the information about the number of open degrees of freedom and optionally on the moved-out position of the patient couch. Advantageously the information about the at least one constructionally predetermined degree of freedom is available to the evaluation apparatus. This can make it possible for the evaluation apparatus to calculate the position of the object especially precisely, in particular even when magnetic field data is available from only relatively few magnetic field sensors. The information can for example be held in the form of configuration files on the system and/or in the evaluation apparatus. For example the evaluation apparatus can be embodied to access the information that is stored on a data storage medium and/or a server. The object ID can also be referred to as the object identification means. The object ID is in particular embodied so that the evaluation apparatus, with the help of the object ID, can assign the information to the respective object, in particular when the information is stored on the evaluation apparatus. The information and optionally the object ID does not have to be transferred to the evaluation apparatus at the same time as the magnetic field measurement data. What is relevant is that this information is available for the evaluation apparatus during determination of the object position data. In particular there is provision for the evaluation apparatus also to have information about a relative position of the at least one magnetic field sensor on the object or for this information to be transferred to the evaluation apparatus and for the evaluation apparatus to take account of this position when calculating first object position data. In particular the position of the at least one magnetic field sensor on the object is fixedly defined.

In a further method step the patient couch with the positioned object is moved into the patient tunnel. Accordingly the moved-in position or the change of position of the patient couch is transferred to the evaluation apparatus, which calculates second object position data from the transferred moved-in position or change of position of the patient couch and based on the first object position data, which specifies the position of the movable object relative to the hybrid system. The order of the steps can be varied in part. In particular the calculation of the first and second object position data can also be provided directly after one another. Also for example the first object position data can be provided merely as an intermediate step and does not have to be explicitly retained as a result. Relevant in particular is the result of the second object position data. Subsequently, from the second object positions data, optionally with the help of the first object position data, the position of the PET attenuation map is determined, in particular by the second object position data being applied to a table offset matrix. A table offset matrix is generally in particular embodied to translate a position of the patient couch to the PET system or the PET detector. In particular the table offset matrix defines a difference of the position between the coordinate system of the patient couch and the coordinate system of the PET system or of the PET detector. In particular in this way a section of the PET attenuation map can be established, which, as regards the moved object, is relevant for the subsequent image correction. This section is forwarded to the computer carrying out the image reconstruction. The position of the PET attenuation map is determined in particular relative to the patient couch and/or relative to the main magnet of the hybrid system. The advantage of this method is that, via the reference measurement from the moved-out position of the patient couch, and taking into account the moved-in position of the patient couch, a precise and automated position determination of the movable object is possible, which has been carried out previously as a rule by a user manually with a time-consuming method, in which typically a PET image and relative thereto an attenuation map has had to be moved manually until the two were congruent.

In accordance with one form of embodiment the movable object is a phantom, in particular a PET phantom, a phantom holder, a local MR receive coil, in particular an MR knee coil or an MR breast coil, or a part of the body. The type of the movable object can in particular define how many open degrees of freedom the movable object has. Since phantoms, through their embedding in phantom holders, are preferably located in the horizontal location on the patient couch, they tend to have fewer degrees of freedom by comparison with the local MR coils and the position determination is made easier in particular by the fact that the measurement signal of the Hall sensor does not have to be evened out by way of an additional acceleration sensor. For the position determination of phantoms it is desirable as a rule that, to calculate the attenuation map, an especially good accuracy of the position determination is achieved. This can be achieved with the inventive method, in that both the constructionally determined or predetermined degrees of freedom and also the data of the at least one magnetic field sensor, preferably of a number of magnetic field sensors, is taken into account. In the area of the local MR receive coils there can be differences as regards the number of the open and the fixed degrees of freedom. A few MR receive coils, in particular the knee and the breast coil, can be able to be fixed permanently to the patient couch and positioned so that they can only be moved into the patient tunnel, or be moved in and out, in the same way as the patient couch. Accordingly MR receive coils, in particular the breast coils, in a few cases have only one open degree of freedom and the position determination can be able to be carried out with just one magnetic field sensor. A knee coil typically has two open degrees of freedom, in particular in the X and in the Z direction. For example the knee coil can typically have a lateral fixing on the patient couch, which fixes all three angles (yaw angle, pitch angle and roll angle), in particular fixes them to 0°. Yaw angle, pitch angle and roll angle can be referred to as orientation angles. The yaw angle can also be referred to as yawing angle. The yaw angle in particular refers to a rotation about the vertical axis (Y axis). The roll angle in particular refers to a rotation about a longitudinal axis (Z axis). The pitch angle can also be referred to as the angle of pitch. The pitch angle refers in particular to a rotation about a transverse axis (X axis). Typically the knee coil additionally has a linear guide along the X axis, which allows the coil to be moved in the X direction without one of the angles being changed. For example a position determination for the knee coil can be provided with two magnetic field sensors. The position determination of multidimensional MR coils such as the shoulder coil, which are wound around the shoulder of the patient and as a consequence can find themselves partly in a tilted position relative to the patient couch or to the B0 main magnet, are comparatively more complex and as an end result often less precise. A similar problem can arise in the position determination of parts of the body of the patient, since these too do not lie completely flat on the patient couch and in particular is dependent on the posture of the part of the patient's body, which can encourage a tilted position.

In accordance with one form of embodiment a position of the movable object is constructionally rigidly predetermined in the Y direction, so that in particular, as a maximum, only the X and/or Z position is to be determined. In particular the orientation angles, in particular all three orientation angles, of the object can be constructionally fixed. With respect to the position determination of knee coils it can be of importance for example to determine their position in the X and Z direction, since they are able to be positioned in the X and Z direction. In accordance with one form of embodiment a position of the movable object in the X-Y plane is constructionally rigidly predetermined, so that in particular only the Z position is to be determined. In respect of the position determination of breast coils it is in particular of importance to determine their position in the Z direction, since they are only moved in and out of the patient tunnel along with the patient couch and thus are only able to be positioned in the Z direction. The X and/or Y coordinates and also all three orientation angles (roll, pitch, yaw) are fixed (for example 0°) are often constructionally fixed, whereby the position determination is simplified in measurement terms. The preferred position measurement in the Z direction is likewise relevant in the measurement of phantoms, which are often fixed horizontally in phantom holders on the patient couch in the X-Y plane and are only movable in their Z position along with the patient couch. A Z position can be able to be determined quite easily, since the magnetic field outside of the patient tunnel or the main magnet in particular decreases as the Z distance decreases. Furthermore, it is just the Z position relative to the patient couch that is often undefined.

In accordance with one form of embodiment a plurality of magnetic field sensors, in particular a number of at least 3 magnetic field sensors, are provided. Preferably the position determination is carried out via Hall sensors, especially preferably via 3D Hall sensors. There can in particular be provision for the number of Hall sensors to lie between 3 and 8. 3D Hall sensors can relatively easily guarantee a position measurement with good accuracy. For movable objects with more than one open degree of freedom, in particular a number of magnetic field sensors, in particular at least one magnetic field sensor per open degree of freedom, can be provided for position determination. Such movable objects can preferably be local MR coils that are not able to be fixed onto the patient couch in the X and/or Y direction. Phantoms, which are usually measured embedded in a phantom holder and typically demand a higher accuracy in the measurement can likewise be provided. For an especially precise position determination of a phantom, in particular a PET phantom, at least 3 magnetic field sensors can be provided. At least 3 magnetic field sensors can also be provided when a phantom supported on the patient couch, in particular horizontally, is provided. For example a non-fixable phantom, which is/will be placed on the patient couch, typically has three fixed or constructionally predetermined degrees of freedom, in particular the Y coordinate, which corresponds in particular to the height of a surface of the patient on which it is laid, and two angles of rotation (roll and pitch angle), which in particular amount to 0°. Open or non-constructionally predetermined degrees of freedom are in particular the coordinates X and Z as well as the yaw angle about the vertical Y axis. There can optionally be provision that, for the measurement of the magnetic field measurement data, more magnetic field sensors are used and/or that the movable object comprises more magnetic field sensors than the object has non-constructionally predetermined degrees of freedom. In this case a deliberate surplus of magnetic field sensors can also be of advantage in respect of redundancy, since a measurement would still be able to be carried out on failure of a sensor and/or since, by the verification with more magnetic field measurement data, an even greater accuracy can be achieved. For example an even greater accuracy can be achieved by simple means via the sensor data or by a fusion of the sensor data via a Kalman filtering.

In accordance with one form of embodiment a plurality

of magnetic field sensors, in particular a number of at least 3 magnetic field sensors, is provided, wherein in particular at least 2, preferably at least 3, of the plurality of magnetic field sensors are arranged in the object so that at least 2 of their location coordinates, in particular each of their 3 coordinates, is different. In particular there can be provision for at least 2 of the plurality of magnetic field sensors to be arranged in the object so that each of their 3 location coordinates is different or that at least 3 of the plurality of magnetic field sensors are arranged in the object so that at least 2 of their 3 location coordinates are different. As an alternative or in addition there can be provision for the magnetic field sensors to not all lie on a common plane, which is spanned by only two coordinate directions of a coordinate system of the hybrid system and/or of the main magnet of the hybrid system. In other words there can in particular be provision, as an alternative or in addition, for the magnetic field sensors not all to lie on a common plane suitable for a positioning intended for the use of the object to be inclined in at least one direction, i.e. in particular to be positioned neither exactly horizontally nor exactly vertically. In particular the magnetic field sensors can lie on a plane, which is tilted compared to the X-Z plane and/or compared to a horizontal plane. With this form of embodiment a specific and thereby in particular more accurate determination of the position of the object can be made possible.

In accordance with one form of embodiment at least 3 of the plurality of magnetic field sensors are arranged in the object so that each of their 3 spatial coordinates is different and/or that the magnetic field sensors do not all lie on a common plane, which is spanned by only two coordinate directions of a coordinate system of the hybrid system and/or of the main magnet of the hybrid system. The spatial coordinates relate in this case in particular to the coordinate system of the main magnet. Such an arrangement of the magnetic field sensors can make possible an especially great accuracy of the position determination. In particular with this classification a unique allocation of the position with the aid of the magnetic field measurement data will be made possible, since with such an arrangement an equation system without linear dependencies can be set up. In the position measurement of phantoms in phantom holders the magnetic field sensors are preferably arranged in the phantom holder, so that the measurement signal is not additionally attenuated more by the phantom holder. In order to increase the precision in each axis direction, it is advantageous for not all magnetic field sensors to be located just on one straight line, but to span a virtual tilted plane in the phantom holder or in the moved object. This can be made possible by at least 3 of the plurality of magnetic field sensors being arranged so that each of their 3 spatial coordinates is different. Furthermore, it has an advantageous effect on the measurement when the magnetic field sensors are spaced as far apart as possible from one another in the X-Y-Z coordinate system. In particular there can be provision for the magnetic field sensors to be arranged both within the object and also at a maximum average distance from one another. This is achieved in particular when the magnetic field sensors are arranged in the corners of the phantom holder. Optionally, in the case of a cuboid phantom holder, 8 magnetic field sensors are provided, pairs of which each have the same X and Z coordinates but are arranged vertically above one another in the Y direction.

Preferably the magnetic field measurement data is transmitted wirelessly to the evaluation apparatus. In accordance with one form of embodiment the object comprises a radio transmitter and/or an optical transmitter, in particular infrared transmitter, wherein the magnetic field measurement data is transmitted wirelessly with the radio transmitter and/or the optical transmitter to the evaluation apparatus. Preferably a radio transmitter and/or an optical transmitter, in particular an infrared LED, is used for wireless transmission. A wireless transmission has the advantage that typically no choke baluns are required, which would represent further sources of interference, which would have to be corrected by a PET attenuation map. With the optical transmitter in particular an optical transmission of the magnetic field measurement data is possible. The optical transmitter or the term “light” is to be understood in broad terms in this context, light in this sense in particular also comprises infrared and UV light unless otherwise specified. A transmission with an optical transmitter has the advantage that interference with other wireless transmissions can be excluded. In this case a transmission with infrared can be especially reliable. A transmission by LED can be especially energy-saving and be possible with relatively low constructional outlay. The hybrid system preferably comprises a receiver, which is arranged so that an optical signal of the optical transmitter can be received when the object is arranged on the patient couch as intended and the patient couch is moved out. A corresponding receiver, in particular comprising a radio sensor and/or an optical sensor, can be provided, which is embodied to receive the signals from the radio transmitter and/or the optical transmitter. The optical sensor can for example comprise an infrared photo transistor. The receiver can be part of the evaluation apparatus or be connected to the evaluation apparatus and be embodied to forward received data to the evaluation apparatus.

In accordance with one form of embodiment the object comprises a rechargeable battery for supplying energy to the at least one magnetic field sensor, and optionally to the radio transmitter and/or the optical transmitter. The power is supplied in particular to the magnetic field sensors, the radio transmitter and/or the optical sensors via the rechargeable battery. The rechargeable battery can be arranged on or attached to the phantom holder or the movable object. In particular the mobile operation of the measurement apparatus is made easier by this.

In accordance with one form of embodiment the object comprises solar cells, in particular a solar module with solar cells, for charging the battery and/or a charging connection for a charging station for charging the battery. For completely autonomous operation the rechargeable battery can be charged via a solar cell integrated into the phantom holder or the movable object. For example the hybrid system can comprise a light source for irradiating the solar cells. The light source for irradiating the solar cells can be embodied so that it can supply the cells with light when the object is arranged on the patient couch, in particular in the intended position, and the patient couch is in particular moved out of the patient tunnel. The flexibly possible alternative is charging of the battery via a charging connection with an external charging station. There can be provision for charging the rechargeable battery before it is used or before carrying out the other method steps of this method. For example the hybrid system can comprise a charging station for the rechargeable battery outside of the main magnet and/or the rechargeable battery can be charged at a charging outside of the main magnet.

In accordance with one form of embodiment at least one configuration file is held in the evaluation apparatus and/or the evaluation apparatus has access to the at least one configuration file, wherein the configuration file comprises in each case a link between predetermined object IDs of defined objects and predetermined information about at least one constructionally predetermined degree of freedom of the respective defined object in each case, wherein the object ID of the object is transferred to the evaluation apparatus and the evaluation apparatus, based on the object ID and the configuration file, establishes the information about the at least one constructionally predetermined degree of freedom. In particular the information is held in the form of configuration files on the system and/or in the evaluation apparatus. The object ID can also be referred to as the object identification means. With the help of the object-ID by application of the configuration file, the evaluation apparatus can assign the current information to the respective object. The information can be assigned for example via a reconciliation of ID tags. In particular the object ID can comprise at least one ID tag. Thus the evaluation unit can determine in an especially simple and reliable way which object is currently involved and/or which degrees of freedom are exactly constructionally fixed.

A further aspect is an assignment system for positional assignment of a Positron Emission Tomography attenuation map, PET attenuation map, of a movable object, in particular of magnetic resonance tomography accessory parts, relative to a movable patient couch of an MR-PET, comprising a movable object as described herein and an evaluation apparatus as described herein, wherein the assignment system is embodied to carry out a method as described herein. In order to carry out the described method for position determination of movable objects relative to the patient couch or to the B0 main magnet of a positron emission tomography device, in particular the magnetic field measurement data of the at least one magnetic field sensor arranged on the object, in particular on the phantom holder, is transmitted to an evaluation apparatus, which reconciles it with the reference data of the B0 stray field and calculates from this the position of the object, in particular of the phantom holder. A radio transmitter or an optical transmitter can be used for transmission, which are embodied to transfer the magnetic field measurement data wirelessly to the evaluation apparatus and/or to receive it. Moreover, the assignment system can comprise a light source, which explicitly supplies the solar cells via which the rechargeable battery can be charged, with light. All advantages and features of the method can be transferred by analogy to the assignment system and vice versa.

A further aspect is a hybrid system for magnetic resonance tomography and Positron Emission Tomography, MR-PET, wherein the MR-PET comprises a movable patient couch, a patient tunnel and an assignment system as described herein. In general the method for positional assignment of a PET attenuation map of a movable object can be applied. All advantages and features of the method can be transferred by analogy to the hybrid system and vice versa. Accordingly all features and advantages of the method for position determination can be adapted to the hybrid system.

FIG. 1 shows a flow diagram of a method for positional assignment of a PET attenuation map of a movable object 10 in accordance with one form of embodiment of the invention. Optionally, a battery of the object can be charged at the beginning, for example via a charging station or via solar cells on the object. The rechargeable battery can in particular be used to supply at least one magnetic field sensor with energy and/or to make possible wireless data transmission from the object. Then, first of all, a movable object 10 is positioned within a stray magnetic field of the B0 main magnet 20 on a patient couch 120 outside of a patient tunnel 140. Thereafter, magnetic field measurement data 26 is recorded with the at least one magnetic field sensor, which is based on the stray field of the B0 field of the B0 main magnet 20. The evaluation proceeds in this case via the evaluation apparatus 28, which receives the magnetic field measurement data 26 in particular via an optical transmitter 46 and/or a radio transmitter and calculates from it the relevant section 22 of the PET attenuation map for the subsequent image reconstruction via the position of the object 10 in relation to the B0 main magnet 20 in the moved-out state and to the patient couch 120 in the moved-in state and/or determines the position of the PET attenuation map. To this end magnetic field measurement data 26 is transferred to the evaluation apparatus 28 via the optical transmitter 46 and/or the radio transmitter, which in the moved-out state of the patient couch 120, on which the object 10 has been positioned, has been measured by a plurality of magnetic field strength sensors 24. The evaluation apparatus 28 reconciles this magnetic field measurement data 26 with B0 reference data and from it calculates the position of the object 10 relative to the B0 main magnet 20. To do this, the evaluation apparatus 28 also uses information about degrees of freedom of the object on the patient couch, which are defined by the predetermined construction.

The predetermined construction is in particular held as information, for example in the form of configuration data, on the evaluation apparatus or the system and/or can be retrieved from the evaluation apparatus, for example from a data storage unit and/or a server. An assignment of the respective information or information about the current phantom (or the phantom holder) and/or about the MR coil can be undertaken for example via a reconciliation of ID tags (for example object ID, coil ID, phantom ID), which on the one hand are held in the configuration files or on the other hand are transferred from the object, in particular from a transmitter, to the system. This enables the evaluation unit in particular to determine which object is involved and which degrees of freedom are fixed at which values. The ID tags can in particular be supplemented with information about (constructionally predetermined and free) degrees of freedom. Corresponding information can for example be designated in the following way:
Object ID=“knee coil”, Fixed_DegreeOfFreedom=“Yaw=0°, Pitch=°, Roll=0°, DCS. Y=−200 mm”.
Object ID=“breast coil”, Fixed_DegreeOfFreedom=“Yaw=0°, Pitch=°, Roll=0°, DCS. Y=−200 mm, DCS. X=0mm”.
Object ID=“sphere phantom-2”, Fixed_DegreeOfFreedom=“Pitch=°, Roll=0°, DCS. Y=−200 mm”.

Based on this first object position data 12 and measurement data (second object position data) 27 about the position of the patient couch 120 in the moved-in state of the patient couch 120, second object position data 13 is established by the evaluation apparatus 28, which represents the position of the object 10 relative to the patient couch 120. The section 22 of the PET attenuation map that is relevant for the image reconstruction is selected and/or the position of the PET attenuation map is established from this second object position data 27 via a table offset matrix. This section 22 is forwarded to a computer which carries out the attenuation correction.

FIG. 2 shows a perspective view of one form of embodiment of an object 10, namely of a phantom holder, with three magnetic field strength sensors 24. Using the phantom holder an, in particular spherical, phantom can be held in a phantom holder pocket 18 for carrying out the positioning method as described herein, in particular as described in relation to FIG. 1, and positioned with the phantom holder on the patient couch 120 so that it is rigidly supported horizontally in the X-Y plane and is only able to be moved in a similar way to the patient couch 120 in the Z direction together with the phantom holder. A fixing of the X position and of the yaw angle can in particular be predetermined by a recess in the patient couch and its side edges. Thus only one open degree of freedom is produced, so that the measurement for enhanced claims of accuracy in the position determination of phantoms with phantom holders with three magnetic field strength sensors 24 is able to be carried out precisely. In this form of embodiment the three magnetic field strength sensors 24 span a virtually inclined plane in the phantom holder. The three magnetic field strength sensors 24 can, unlike in this form of embodiment, preferably be arranged in the phantom holder so that each of their three spatial coordinates is different. It is in particular advantageous for two or more of the magnetic field strength sensors 24 to be positioned in the corners of the phantom holder, so that for further increasing the measurement accuracy, the magnetic field strength sensors 24 are arranged at a maximum distance from one another. The aim of such an arrangement is in particular, with a minimum number of magnetic field strength sensors 24 where possible, to achieve a maximum measurement accuracy.

FIG. 3 shows a perspective view of one form of embodiment of a phantom holder with eight magnetic field strength sensors 24. Similarly to FIG. 2, the phantom is positioned via a phantom holder on the patient couch 120 in order to restrict the open degrees of freedom to the Z direction. In this exemplary embodiment, for an optimum measurement accuracy, eight magnetic field strength sensors 24 have been positioned so that all corners of the phantom holder are covered and thus the magnetic field strength sensors 24 are spaced at the maximum distance from one another. In this arrangement four pairs of magnetic field strength sensors are produced that have the same X and Z coordinates but are arranged vertically one above another in the Y direction. The transmission of the measurement data (and preferably of an object ID) is undertaken in this exemplary embodiment via an optical transmitter 46, which transfers the magnetic field measurement data 26/27 to the evaluation apparatus 28. The optical transmitter 46 can in particular be an IR transmit diode. In order to guarantee an autonomous operation of the magnetic field strength sensors 24 and of the optical transmitter 46, these are supplied with power via a rechargeable battery 42, which is able to be charged via a solar cell 44. As an alternative the rechargeable battery 42 can be charged via a charging station.

FIG. 4 shows a perspective view of one form of embodiment of a hybrid system for magnetic resonance tomography and positron emission tomography. The method and the assignment system for positional assignment of a PET attenuation map of a movable object is preferably applied during the MR-PET, since in this medical imaging method a high accuracy and a precise determination of the position the PET attenuation map is required. Shown in FIG. 4 is a magnetic resonance positron emission tomography system 100 with a moved-out patient couch 120, on which a movable object 10, in the form of a PET phantom, or of a phantom holder for a PET phantom, is located. In this state it is possible to establish magnetic field measurement data 26 for position determination of the object 10 or in relation to the B0 main magnet 20 with an evaluation apparatus 28. This magnetic field measurement data 26 is transmitted to the evaluation apparatus 28 wirelessly and/or by an optical transmitter 46. Measurement data 27 for the position of the patient couch 120 is transmitted to the evaluation apparatus 28 when the patient couch 120 with the object 10 or with the patient is moved into the patient tunnel 140. From the magnetic field measurement data 26 and the measurement data 27 about the position of the patient couch, first and second object position data 12/13 is established by the evaluation apparatus 28, from which the section 22 of the PET attenuation map relevant for the image reconstruction is determined via a table offset matrix and transferred to a further computer.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for positional assignment of a Positron Emission Tomography (PET) attenuation map of a movable object, relative to a movable patient couch of a magnetic resonance tomography (MR)-PET system, wherein the MR-PET system comprises the movable patient couch and a patient tunnel with a B0 main magnet, at least one of a position or an orientation of the object is constructionally predetermined with respect to at least one degree of freedom and is free with respect to at least one further degree of freedom, the object comprises at least one magnetic field sensor for each non-predetermined degree of freedom at the respective relative position, the method comprising:
positioning the movable object within a stray magnetic field of the B0 main magnet on the patient couch, when the patient couch is positioned outside of the patient tunnel;
recording magnetic field measurement data with the at least one magnetic field sensor and transferring the magnetic field measurement data to an evaluation apparatus, wherein information about the at least one constructionally predetermined degree of freedom is transferred to the evaluation apparatus or the magnetic field measurement data is stored on the evaluation apparatus;
calculating first object position data of the object relative to at least one of the patient couch or the main magnet with the evaluation apparatus based on the magnetic field measurement data;
moving the patient couch with the movable object into the patient tunnel and transferring at least one of the moved position or a change in position of the patient couch to the evaluation apparatus;
calculating second object position data relative to the B0 main magnet with the evaluation apparatus based on the first object position data and at least one of the moved-in position or the change in position of the patient couch; and
at least one of determining the position of the PET attenuation map or selecting a section of the PET attenuation map based on the second object position data.

2. The method of claim 1, wherein the movable object is a phantom, a phantom holder, a local MR receive coil, or a part of a body.

3. The method of claim 1, wherein a position of the movable object, at least in a Y direction, is constructionally rigidly predetermined such that only at least one of an X or a Z position is to be determined as a maximum.

4. The method of claim 1, wherein
the at least one magnetic field sensor is a plurality of magnetic field sensors, and
at least 2 of the plurality of magnetic field sensors are in the object such that at least one of at least 2 of spatial coordinates of the magnetic field sensors is different or that the magnetic field sensors do not all lie on a common plane.

5. The method of claim 1, wherein
the object comprises at least one of a radio transmitter or an optical transmitter, and
the magnetic field measurement data is transmitted wirelessly with the at least one of the radio transmitter or the optical transmitter to the evaluation apparatus.

6. The method of claim 5, wherein the object comprises a rechargeable battery to supply energy to the at least one magnetic field sensor.

7. The method of claim 6, wherein at least one of
the object comprises solar cells configured to charge the rechargeable battery, or
the object comprises a charging connection for a charging station to charge the rechargeable battery.

8. The method of claim 1, wherein
at least one of at least one configuration file is stored in the evaluation apparatus or the evaluation apparatus has access to the at least one configuration file,
the configuration file comprises a link between predetermined object identifications (IDs) of defined objects each with predetermined information about at least one constructionally predetermined degree of freedom of the respective defined object in each case,
the object ID of the object is transferred to the evaluation apparatus and the evaluation apparatus, based on the object ID and the configuration file.

9. An assignment system for positional assignment of a Positron Emission Tomography (PET) attenuation map of a movable object relative to a movable patient couch of a magnetic resonance tomography (MR)-PET system, wherein the assignment system is configured to perform the method of claim 1.

10. A hybrid system for magnetic resonance tomography (MR) and Positron Emission Tomography (PET) comprising:
a movable patient couch;
a patient tunnel; and
the assignment system of claim 9.

11. The method of claim 1, further comprising:
transferring the position of the patient couch to the evaluation apparatus.

12. The method of claim 1, wherein the calculating calculates the first object position data further based on the position of the patient couch and on the information about the at least one constructionally predetermined degree of freedom.

13. The method of claim 4, wherein the plurality of magnetic field sensors is at least 3 magnetic field sensors.

14. The method of claim 6, wherein the rechargeable battery is configured to supply energy to the at least one of the radio transmitter or the optical transmitter.

15. The method of claim 2, wherein a position of the movable object, at least in a Y direction, is constructionally rigidly predetermined such that only at least one of an X or a Z position is to be determined as a maximum.

16. The method of claim 15, wherein
the at least one magnetic field sensor is a plurality of magnetic field sensors, and
at least 2 of the plurality of magnetic field sensors are in the object such that at least one of at least 2 of spatial coordinates of the magnetic field sensors is different or that the magnetic field sensors do not all lie on a common plane.

17. The method of claim 16, wherein
the object comprises at least one of a radio transmitter or an optical transmitter, and
the magnetic field measurement data is transmitted wirelessly with the at least one of the radio transmitter or the optical transmitter to the evaluation apparatus.

18. The method of claim 17, wherein the object comprises a rechargeable battery to supply energy to the at least one magnetic field sensor.

19. The method of claim 18, wherein at least one of
the object comprises solar cells configured to charge the rechargeable battery, or
the object comprises a charging connection for a charging station to charge the rechargeable battery.
